(19) United States
(12) Patent Application Publication
(10) Pub. No.: US 2004/0181227 A1
(43) Pub. Date: Sep. 16, 2004

(54) System and method for attaching a bone plate to bone

(76) Inventor: Farid Khalili, Briar Cliff Manner, NY (US)

Correspondence Address: Eric A. LaMorte, LaMorte & Associates, P.C., P.O. Box 434, Yardley, PA, 19067-8434, US

(21) Appl. No.: 10/387,110

(22) Filed: Mar. 11, 2003

(51) Int. Cl. A61F 2/30

(52) U.S. Cl. 606/69

(57) ABSTRACT

A system and method for attaching a bone plate to bone using specialized bone screws. A bone plate is provided that contains at least one screw aperture. The wall that defines the screw aperture is torically curved. A similarly torically curved collar element is inserted into each screw aperture. Bone screws are provided. Each bone screw has a top end, a bottom end and external threads that extend between the top end and the bottom end. The threads have a constant pitch and thread diameter between the first end and the second end. However, each bone screw has a shaft diameter that increases from its bottom end to its top end. As the coil element advances on the bone screw, its diameter increases. Eventually, the coil element engages the collar element and expands the collar element.

### BACKGROUND OF THE INVENTION

1. Field of the Invention

The present invention relates to bone plates and the screws that are used to attach bone plates to bone. More particularly, the present invention relates to systems that use bone plates with screws that can be inserted into the bone at a variety of angles relative the bone plate.

2. Description of Related Art

Bone plates are used to rigidly bind two sections of bone together. Although bone plates are used by surgeons to strengthen and/or repair a variety of bones, bone plates are commonly used on the cervical portion of the spine to fuse together adjacent vertebrae. When bone plates are attached to vertebrae, the bone plates are mounted to the bone of the vertebrae using bone screws. As such, the bone plate is typically manufactured with screw apertures through which the bone screws can pass.

When attaching a bone plate to vertebrae, bone screws can be passed either completely through the vertebrae, using bi-cortical screws, or partially through the vertebrae, using uni-cortical screws. Each method has its advantages and disadvantages. By passing a bi-cortical bone screw completely through the vertebra, a strong mounting attachment is made. Since the spine experiences a great deal of stress, twisting and turning, a strong mounting attachment is needed to ensure that the bone screws do not disengage from the bone and the bone plate become loose. However, the disadvantage of passing a bi-cortical bone screws completely through the vertebrae is that the protruding section of the screw passes into soft tissue that surrounds the vertebrae. As such, the protruding bone screw may result in a variety of different medical complications. Some complications may result in temporary of permanent paralysis.

To avoid the possibility of such severe complications, many surgeons elect to use uni-cortical bone screws that do not pass through the vertebrae. By using uni-cortical bone screws, the screws partially penetrate the vertebrae and terminate within the vertebrae. Since uni-cortical bone screws do not pass through the vertebrae, they engage less bone than do bi-cortical bone screws. Consequently, uni-cortical screws tend to be less strong than bi-cortical screws.

When first used, uni-cortical screws were inserted into the vertebrae at a perpendicular angle, the same way a wood screw is inserted into a piece of wood. However, it was soon learned that the strength of the uni-cortical bone screws can be improved by inserting the bone screws into the vertebrae at various diverging and converging angles. As such, many bone plates were created with angled screw apertures to accommodate the angled insertion of the uni-cortical bone screws.

As all surgeons know, there are anatomical differences between all people. As such, the position, size and structure of the vertebrae vary from patient to patient. Accordingly, the appropriate angles for inserting a uni-cortical bone screw into a particular patient's vertebrae also varies from patient to patient. The use of bone plates with bone screws that can only be inserted at one angle often causes a surgeon to compromise during the implantation of that bone plate. This compromise results in bone screws that are implanted at less than ideal angles and at less than ideal positions.

U.S. Pat. No. 6,030,389 to Wagner et al., entitled, System And Method For Stabilizing The Human Spine With A Bone Plate, discloses a system where uni-cortical bone screws can be inserted through a bone plate at a variety of different angles. However, in the system described in the Wagner patent, the head of the bone screws are very finely threaded. The bone screws are also implanted at a variety of angles. Under such circumstances it is not uncommon to have a cross-threading condition occur, or to have debris catch in the threads. If either condition occurs, a surgeon often turns the bone screw tighter to work through the obstruction rather than reverse the screw to eliminate the obstruction. As a result, many surgeons over tighten the bone screws and strip the bone surrounding the bone screw, when trying to advance the bone screw properly through the bone plate.

A need therefore exists for an improved system and method for attaching a bone plate to bone with screws, wherein the bone screws can be inserted into the bone at a variety of angles and has an improved bone plate engagement design that minimizes the possibility of over tightening by a surgeon. This need is met by the present invention as described and claimed below.

### SUMMARY OF THE INVENTION

The present invention is a system and method for attaching a bone plate to bone using specialized bone screws. A bone plate is provided that contains at least one screw aperture. The wall that defines the screw aperture is torically curved. A similarly torically curved collar element is inserted into each screw aperture. Since the collar element and the screw aperture are similarly curved, the collar element can move in the screw aperture in a manner similar to a ball in a ball and socket joint. The center of the collar elements are open.

Bone screws are provided. Each bone screw has a top end, a bottom end and external threads that extend between the top end and the bottom end. The threads have a constant pitch and thread diameter between the first end and the second end. However, each bone screw has a shaft diameter that increases from its bottom end to its top end.

A coil element is placed around each bone screw, wherein the coil element lay between the threads on the exterior of the screw. As the bone screw is advanced into the collar element and the screw aperture, the coil element advances up the exterior of the bone screw. As the coil element advances, its diameter increases. Eventually, the coil element engages the collar element and expands the collar element until it abuts against the interior of the screw aperture. This locks the collar element and coil element into place. As such, the bone screw can firmly engage the bone plate without the use of a threaded connection.

### BRIEF DESCRIPTION OF THE DRAWINGS

For a better understanding of the present invention, reference is made to the following description of exemplary embodiments thereof, considered in conjunction with the accompanying drawings, in which:

FIG. 1 is an exploded perspective view of an exemplary embodiment of the present invention system;

FIG. 2 is an exploded cross-sectional view of the system shown in FIG. 1;

FIG. 3A is a cross-sectional view of a coil element engaging the lower section of a bone screw;

FIG. 3B is a cross-sectional view of a coil element engaging the upper section of a bone screw; and

FIG. 4, shows a cross-sectional view of the present invention system after installation.

### DETAILED DESCRIPTIONS OF THE DRAWINGS

Although the present invention can be used with most any type of bone plate for use in binding any type of bone, the present invention is particularly well suited for use in spinal bone plates for use in binding separate vertebrae. Accordingly, by way of example, the present invention system and method will be described in use on the spine, in order to set forth the best mode contemplated for the invention.

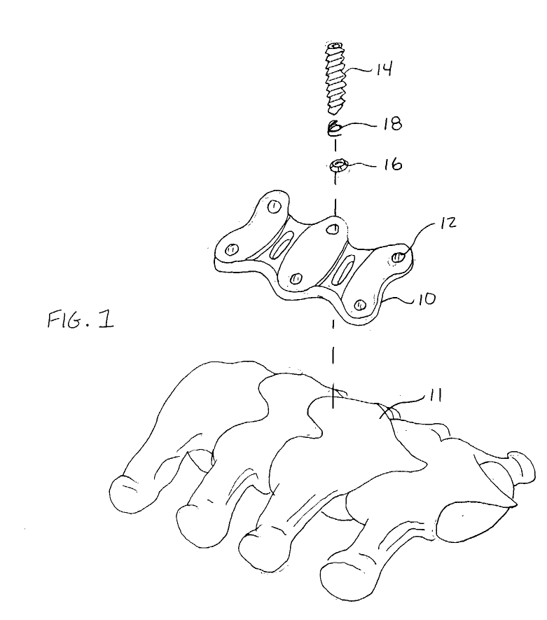

Referring to FIG. 1, a segment of the spine 11 is shown containing various vertebrae. Three of the vertebrae are to be mechanically joined and stabilized using a bone plate 10. The bone plate 10 contains screw apertures 12 that are positioned over the mid-section of each of the vertebrae that are to be bound. There are many different sizes, lengths and shapes of bone plates. The appearance of the bone plate 10 shown in FIG. 1 is merely exemplary. Any known bone plate design can be adapted for use with the present invention, provided that the bone plate design contains screw apertures of the type that are later described.

The bone plate 10 is mounted to the various vertebrae using specialized bone screws 14. However, the bone screws 14 do not directly engage the screw apertures 12 in the bone plate 10. Rather, two interposing elements are used to interconnect the bone screws 14 to the interior surfaces of the screw apertures 12. Those interposing elements include a collar element 16 and a coil element 18.

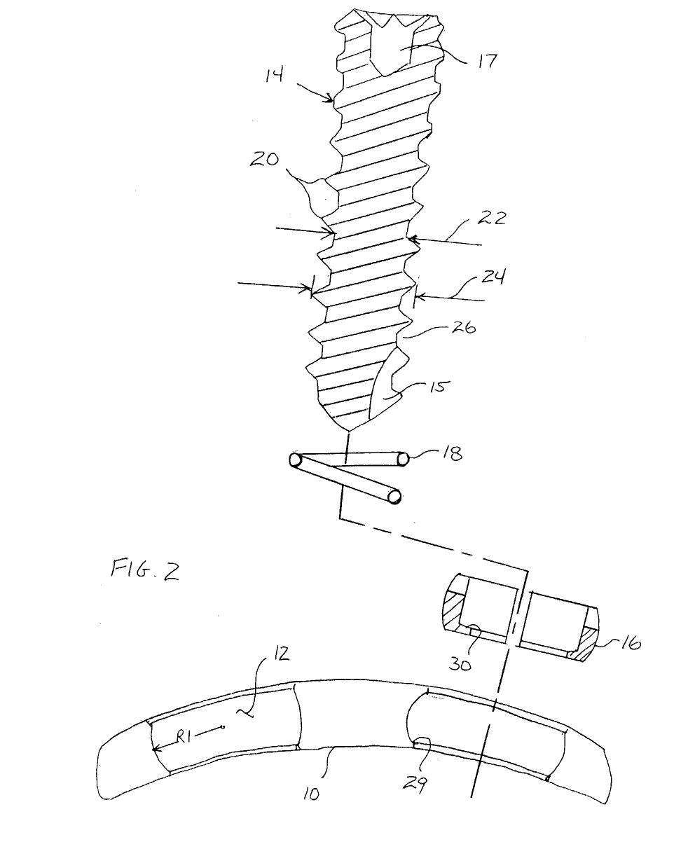

Referring to FIG. 2, it can be seen that the screw apertures 12 in the bone plate 10 are generally circular in shape. However, the interior wall of each screw aperture 12 is torically curved, having a predetermined radius of curvature R1. Due to the torically curved shape of the wall defining the screw aperture 12, the diameter of the screw aperture 12 is wider in the middle of the bone plate 10 than it is at either the top or bottom of the bone plate 10.

From FIG. 2, it can also be seen that a bone screw 14 is passed into the screw aperture 12 on the bone plate 10. It is the bone screw 14 that attaches the bone plate 10 to the bone. Each bone screw 14 has a top end and a bottom end. At the bottom end of the screw is formed a cut relief 15 so that the bone screw 14 will cut into bone. At the top end of the bone screw 14 is formed a shape depression 17 for receiving a driving element, such as an Allen key. Depending upon the type of driver preferred, the shaped depression 17 at the top end of the bone screw 14 can have a square shape. hexagonal shape or star shape.

The bone screw 14 is threaded continuously between its top end and bottom end. Consequently, the bone screw 14 has threads 20, a threaded diameter 22 and a shaft diameter 24. The pitch of the threads 20 on the bone screw 14 remain constant along the entire length of the bone screw 14. Similarly, the threaded diameter 22 of the bone screw 14 remains constant along the full length of the bone screw 14. However, the shaft diameter 24 of the bone screw 14 varies as a function of position along the length of the bone screw 14. At the bottom end of the bone screw 14, the bone screw 14 has a first shaft diameter. However, the shaft diameter 24 increases steadily as it moves from the bottom end of the bone screw 14 toward the top end of the bone screw 14. As a result, by the top end of the bone screw 14, the shaft diameter 24 has increased to a wider second shaft diameter.

Since the bone screw 14 has a constant threaded diameter 22, and a shaft diameter 24 that increases as it moves toward the top end of the bone screw 14, the troughs 26 between the threads 20 become shallower toward the top end of the bone screw 14. Similarly, since the pitch of the threads 20 remains constant, but the shaft diameter 24 increases, the threads 20 become truncated near the top end of the bone screw 14, wherein the threads 20 no longer terminate at a sharp edge.

The threaded diameter 22 of the bone screw 14 is smaller than the interior diameter of the screw aperture 12. As such, the bone screw 14 cannot by itself engage the bone plate 10. A collar element 16 is provided to help the bone screw 14 engage the screw aperture 12. The collar elements 16 are annular in shape and are slotted so they can readily expand. The exterior of the collar elements 16 are torically curved. The radius of curvature R2 exhibited by the exterior of the collar elements 16 matches the radius of curvature R1 exhibited by the wall defining the screw apertures 12. As such, when the collar element 16 is positioned within the screw aperture 12, the collar element 16 functions as a ball in a ball joint. The collar element 16 can therefore move within the confines of the screw aperture 12 within a predetermined range of motion.

At the bottom of the screw aperture 12 is formed a small ledge 29. The presence of the ledge 29 prevents the collar element 16 from being forced through the screw aperture 12 and prevents any portion of the screw aperture 12 from protruding below the bone plate 10.

The interior of the collar element 16 is cylindrical and open. At the bottom of the interior of the collar element 16 is an annular ledge 30. The interior diameter of the annular ledge 30 is just large enough to enable the threads 20 of the bone screw 14 to pass unencumbered.

The collar element 16 is placed into the screw aperture 12. However, the interior structures of the collar element 16 do not engage the bone screw 14. This interconnection is made using a coil element 18. The coil element 18 is a structure shaped like a short segment of a coil spring. The pitch of the coils in the coil element 18 match the thread pitch of the bone screw 14.

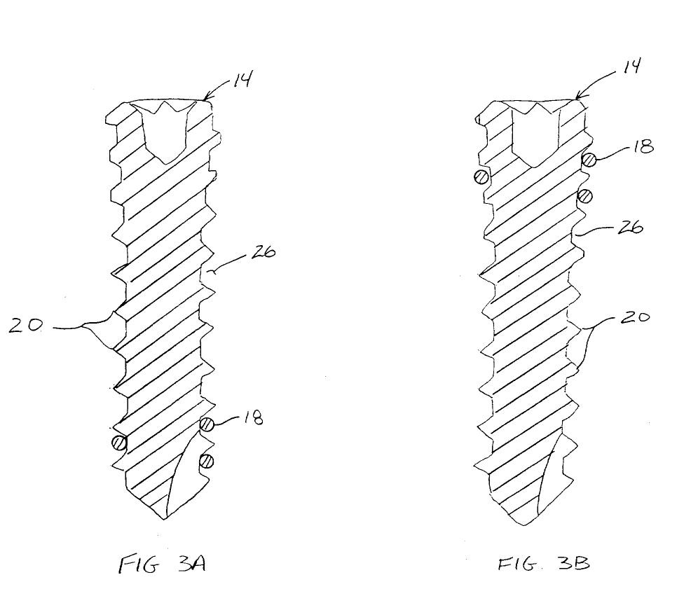

Referring to FIG. 3A, it can be seen that when the coil element 18 is threaded onto the bottom of the bone screw 14, the turns of the coil element 18 pass between the threads 20 on the bone screw 14. As such, the turns of the coil element 18 pass through the troughs 26 on the bone screw 14. On the bottom section of the bone screw 14, the depths of the troughs 26 between the threads 20 are greater than the diameter of the wire comprising the coil element 18. As such, the tips of the screw threads 20 protrude farther than do the turns of the coil element 18. However, referring now to FIG. 3B, it can be seen that as the coil element 18 advances toward the top of the bone screw 14, the troughs 26 between the threads 20 become shallower and the turns of the coil element 18 begin to protrude beyond the threads 22 of the bone screw 14.

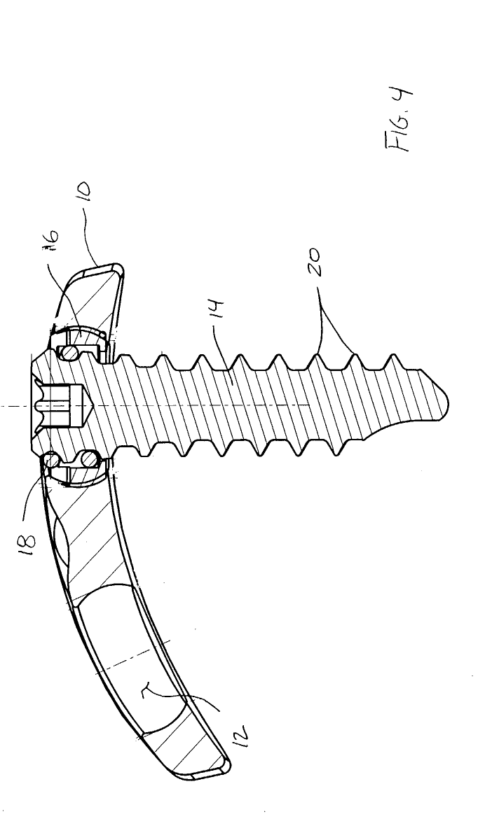

Referring to FIG. 4, it will now be understood that to use the present invention system, the location where the bone plate 10 is to be mounted is selected by a surgeon. Guide holes are then drilled into the bone at positions corresponding to the screw apertures 12 in the bone plate 10. The angle of the guide holes is selected by the surgeon. The bone plate 10 is then placed onto the bone. Collar elements 16 are placed into the screw apertures 12 on the bone plate 10. Coil elements 18 are then placed into the collar elements 16. Bone screws 14 are then advanced through the coil element 18 and the collar element 16 into the bone. The bone screw 14 follows the angle of the guide hole. As the bone screw 14 is advanced, the coil element 18 begins to expand and engages the collar element 16. As the coil element 18 engages the collar element 16, the collar element 16 rotates in the screw aperture 12 of the bone plate 10, so as to be in proper alignment with the advancing bone screw 14. As the top of the bone screw 14 advances to the level of the bone plate 10, the turns of the coil element 18 expand the collar element 16 against the interior of the screw aperture 12 in the bone plate 10. The result is that the bone screw 14, coil element 18, collar element 16 and bone plate 10 become mechanically locked at the selected orientation.

Since the threads 20 on the bone screw 14 engage no other threads, there is no chance of cross-threading during installation. As such, the potential for damage to either the bone screws 14 or the bone plate 10 during installation is eliminated. This results in a more accurate and trouble-free installation procedure.

It will be understood that the embodiment of the present invention system and method described and illustrated are merely exemplary and persons skilled in the art can make many variations to the shown embodiments. For example the shape of the bone plate can be altered to any known configuration. The number of coil turns in the spring element can be altered. The length, thread pitch, pitch diameter, shaft diameter and change in shaft diameter associated with the bone screw can also be varied into configurations not specifically shown. All such modifications and alternate embodiments are intended to be covered by the present invention as claimed below.

What is claimed is:

1. A system, comprising:
a bone plate having at least one screw aperture formed therethrough;
a bone screw for each said screw aperture, wherein each bone screw has a top end a bottom end and threads that extend from said top end to said bottom end;
an annular collar element for each screw aperture, said collar element being sized to be received within a screw aperture, wherein said collar element defines a central opening through which said bone screw can pass;
a coil element for engaging each said bone screw between said threads, wherein said coil element expands and engages said collar element as said bone screw is advanced through said screw aperture in said bone plate, thereby causing said collar element to engage said bone plate.

2. The system according to claim 1, wherein each said screw aperture is defined in said bone plate by a torically curved surface.

3. The system according to claim 1, wherein each said collar element has a torically curved exterior surface that enables said collar element to move within said screw aperture before being biased against said bone plate by said coil element.

4. The system according to claim 1, wherein said bone screw has a constant thread pitch between said top end and said bottom end.

5. The system according to claim 1, wherein said bone screw has a constant thread diameter between said top end and said bottom end.

6. The system according to claim 5, wherein said bone screw has a shaft diameter that increases from said bottom end toward said top end.

7. The system according to claim 6, wherein troughs exist between threads on said bone screw, wherein the depth of said troughs decrease from a maximum depth to a minimum depth as a function of position between said bottom end of said bone screw and said top end of said bone screw.

8. The system according to claim 7, wherein said coil element is comprised of wire having a diameter that is smaller than said maximum depth of said troughs and larger than said minimum depth of said troughs.

9. The system according to claim 1, wherein said central opening of said annular collar contains an annular ledge, wherein said annular ledge prevents said coil element from passing through said central opening.

10. A bone screw, comprising:
a threaded screw having a top end, a bottom end and external threads that extend between said top end and said bottom end,
wherein said threads have a constant pitch and thread diameter between said first end and said second end; and
wherein said threaded screw has a shaft diameter that increases between said bottom end and said top end.

11. The bone screw according to claim 10, further including a formed drive depression in said top end of said threaded screw.

12. A method of affixing a bone plate to a bone, comprising the steps of:
providing a bone plate that defines a plurality of bone screw apertures;
providing bone screws, wherein each bone screw has a top end, a bottom end and external threads that extend between said top end and said bottom end, wherein said threads have a constant pitch and thread diameter between said top end and said bottom end, and wherein said threaded screw has a shaft diameter that increases between said bottom end and said top end;
providing collar elements that are positioned within said screw apertures;
providing coil elements that are positioned within said collar elements;
advancing said bone screws through said coil elements and said collar elements in each screw aperture, wherein said bone screws expand said coil element causing said coil elements to expand said collar elements and engage said bone plate.

13. The method according to claim 12, wherein each said screw aperture is defined in said bone plate by a torically curved surface.

14. The method according to claim 13, wherein each said collar element has a torically curved exterior surface that enables said collar element to move within said screw aperture before being biased against said bone plate by said coil element.

15. The method according to claim 15, further including the step of positioning said collar element in said screw aperture to be aligned with said bone screw.
